SEQUENCE LISTING (21 sequences)

SEQ ID NO: 1 - protein, 62 aa, Naja atra
LECHNQQSSQTPTTTGCSGGETNCYKKRWRDHRGYRTERGCGCPSVKNGIEINCCTTDRCNN

SEQ ID NO: 2 - protein, 83 aa, Naja atra
MKTLLLTLLVVTIVCLDLGYTLECHNQQSSQTPTTTGCSGGETNCYKKRWRDHRGYRTERGCGCPSVKNGIEINCCTTDRCNN

SEQ ID NO: 3 - protein, 62 aa, Naja atra
LECHNQQSSQTPTTTGCSGGETNCYKKRWRDHRGYRTERGCGCPIVKNGIESNCCTTDRCNN

SEQ ID NO: 4 - protein, 74 aa, Bungarus multicinctus
IVCHTTATSPISAVTCPPGENLCYRKMWCDAFCSSRGKVVELGCAATCPSKKPYEEVTCCSTDKCNPHPKQRPG

SEQ ID NO: 5 - protein, 74 aa, Bungarus multicinctus
IVCHTTATIPSSAVTCPPGENLCYRKMWCDAFCSSRGKVVELGCAATCPSKKPYEEVTCCSTDKCNHPPKRQPG

SEQ ID NO: 6 - protein, 72 aa, Naja anchietae
IRCFITPDVTSQACPDGQNICYTKTWCDNFCGMRGKRVDLGCAATCPTVKPGVDIKCCSTDNCNPFPTRERS

SEQ ID NO: 7 - protein, 71 aa, Naja Nivea
IRCFITPDVTSQACPDGHVCYTKMWCDNFCGMRGKRVDLGCAATCPKVKPGVNIKCCSRDNCNPFPTRKRS

SEQ ID NO: 8 - protein, 72 aa, Ophiophagus hannah
TKCYKTGDRIISEACPPGQDLCYMKTWCDVFCGTRGRVIELGCTATCPTVKPHEQITCCSTDNCDPHHKMLQ

SEQ ID NO: 9 - protein, 72 aa, Ophiophagus hannah
TKCYKTGDRIISEACPPGQDLCYMKTWCDVFCGTRGRVIELGCTATCPTVKPHEQITCCSTDNCNPHPKMKQ

SEQ ID NO: 10 - protein, 72 aa, Ophiophagus hannah
TKCYITPDVKSETCPDGENICYTKSWCDVFCTSRGKRIDLGCAATCPKVKPGVDIKCCSTDNCNPFTPWKRH

SEQ ID NO: 11 - protein, 73 aa, Ophiophagus hannah
TKCYVTPDATSQTCPDGQDICYTKTWCDGFCSSRGKRIDLGCAATCPKVKPGVDIKCCSTDNCNPFPTWKRKH

SEQ ID NO: 12 - protein, 73 aa, Ophiophagus hannah
TKCYVTPDVKSETCPAGQDLCYTETWCVAWCTVRGKRVSLTCAAICPIVPPKVSIKCCSTDACGPFPTWPNVR

SEQ ID NO: 13 - protein, 73 aa, Ophiophagus hannah
TKCYVTPDVKSETCPAGQDICYTETWCDAWCTSRGKRVDLGCAATCPIVKPGVEIKCCSTDNCNPFPTWRKRP

SEQ ID NO: 14 - protein, 61 aa, Naja kaouthia
LECHNQQSSQAPTTKTCSGETNCYKKWWSDHRGTIIERGCGCPKVKPGVNLNCCRTDRCNN

SEQ ID NO: 15 - protein, 62 aa, Naja kaouthia
LECHNQQSIQTPTTTGCSGGETNCYKKRWRDHRGYRTERGCGCPSVKNGIEINCCTTDRCNN

SEQ ID NO: 16 - protein, 83 aa, Naja kaouthia; identical to SEQ ID NO: 2

SEQ ID NO: 17 - protein, 60 aa, Dendroaspis polylepis
  misc_feature (1)..(1): Xaa can be any naturally occurring amino acid
XICYNHQSTTRATTKSCEENSCYKKYWRDHRGTIIERGCGCPKVKPGVGIHCCQSDKCNY

SEQ ID NO: 18 - protein, 60 aa, Dendroaspis polylepis
RICYNHQSTTRATTKSCEENSCYKKYWRDHRGTIIERGCGCPKVKPGVGIHCCQSDKCNY

SEQ ID NO: 19 - protein, 72 aa, Dendroaspis polylepis
RTCNKTFSDQSKICPPGENICYTKTWCDAWCSRRGKIVELGCAATCPKVKAGVGIKCCSTDNCNLFKFGKPR

SEQ ID NO: 20 - protein, 64 aa, Bungarus fasclatus
RICLNQQQSTPEDQPTNGQCYIKTDCQNKTWNTHRGSRTDRGCGCPKVKPGINLRCCKTDKCNE

SEQ ID NO: 21 - protein, 62 aa, Bungarus fasclatus
RICLNQQSSEPQTTETCPNGEDTCYNKTWNTHRGSRTDRGCGCPKVKPGINLRCCKTDKCNQ

(19) United States
(12) Patent Application Publication
(10) Pub. No.: US 2024/0041988 A1
(43) Pub. Date: Feb. 8, 2024

(54) Use of elapidae postsynaptic neurotoxin in the treatment of over expression of inflammatory cytokines related diseases

(71) Applicant: Zhejing SHEN, Shanghai (CN)

(72) Inventor: Zhejing SHEN, Shanghai (CN)

(21) Appl. No.: 17/928,935

(22) PCT Filed: Apr. 8, 2021
(86) PCT No.: PCT/CN2021/000072

§ 371 Date: Dec. 12, 2022

(30) Foreign Application Priority Data
Jun. 2, 2020 (CN) 202010509545.0

(51) Int. Cl. A61K 38/46; A61P 29/00

(52) U.S. Cl. CPC A61K 38/465; A61P 29/00; C12Y 301/01004

(57) ABSTRACT

A method for treatment of the diseases related to overexpression of tumor necrosis factor-α (TNF-α) and/or interleukin-1β (IL-1β) of a patient. The method comprises: administering a therapeutically effective dose of elapidae postsynaptic neurotoxin molecules (SEQ ID NOs. 1-21) and a pharmaceutically acceptable carrier. The diseases comprise rheumatoid arthritis, rheumatic arthritis, gouty arthritis, osteoarthritis, traumatic arthritis, ankylosing spondylitis, diabetes, diabetic peripheral neuropathy, diabetic retinopathy, systemic lupus erythematosus, neuropathic pain, cancer pains, myocarditis, pancreatic cancer, and liver cancer. The mature proteins or peptides of the elapidae postsynaptic neurotoxin molecules include any one of the amino acid sequences as shown in SEQ ID NO. 1 to SEQ ID NO. 21, or have the homology of 70% or more to the amino acid sequences as shown in SEQ ID NO. 1 to SEQ ID NO. 21 respectively.

### TECHNICAL FIELD

The present invention relates to a group of elapidae postsynaptic neurotoxin monomer molecules that inhibit or reduce inflammatory cytokines in rat, and can alleviate or treat the diseases associated with excessive expression of inflammatory cytokines, belonging to biochemical and biopharmaceutical area.

### BACKGROUND

The inflammation reaction is a spontaneous and protective immune mechanism when the human body is subjected to exogenous invasion or endogenous lesion, however, excessive inflammatory cytokines due to over inflammation reactions can cause a series of autoimmune diseases, such as rheumatoid arthritis (RA), Crohn's disease, diabetes, multiple sclerosis. ext. If inflammation disseminate to blood, such as sepsis shock syndrome, septicemia and severe wound conditions, those inflammation reactions will be more risky than that of the original inflammation. Therefore, under the normal condition, human body can have a mechanism for inhibiting and regulating the inflammatory reaction, so the inflammatory reaction can be in a balanced state. If the inflammatory response of the body is continuously amplified and out of control, the inflammatory cytokines such as tumor necrosis factor-alpha, (TNF-alpha), interleukin-1 (Interleukin-1, IL-1) can be over produced, and the self-destruction generated can be more serious than the damage caused by direct stimulation of pathogenic factors. [1-6]

In 2000, the research of Lyudmia and trace YKJ in shows for the first time that when immune system activated by inflammation response from organism injures, the immune-stimulation signal can be projected to the vagus nerve nucleus through the central nerve system, and the outgoing vagus nerve fiber is activated and peripheral nerve endings will release acetylcholine and combine with alpha 7 subunits of nicotinic acetylcholine receptors (ALPHA) which located on immune cells, then the intracellular signal will inhibit the release of inflammatory cytokines interleucokin-1, (IL-1) and tumor necrosis factor-alpha to regulate inflammatory response. The way to control inflammatory response process through the pathway of outgoing nerves-acetylcholine-nicotinic-acetylcholine receptor is named “cholinergic anti-inflammatory pathway” [1,7]. This is a self regulation mechanism for inhibition of inflammation response. Research shows the activation of “cholinergic anti-inflammatory pathway” can attenuate the inflammation and heal the related disease. [8-10]

Tumor necrosis factor-alpha (TNF-alpha) is produced from a variety of cell types (including monocytes and macrophages), this cytokine was initially identified according to their ability to induce mouse tumor necrosis. (see Old, L. Science 230: 630-632).

TNF-alpha is the earliest and most important inflammatory intermediate of inflammation process, and can activate neutrophils and lymph cells to increase permeability of vascular endothelial cells, regulate metabolic tissues to promote synthesis and release of other cytokines.

Over expression of tumor necrosis factor-alpha relates to patho-physiology of a series of human diseases such as coma, septicemia, infection, autoimmune disease, transplantation rejection and mediation of anti-graft versus host. (see Beltler, B and Cervi, A (1988) ANNU. REV. Biochem 57: 505 -518; Beutler, B AND Cervi, A (1989) ANNU. REV. IMMUNOL. 7: 625-655; Moeller, A, et al (1990) CytoKine 2:162-169; U.S. Pat. No. 5,231,024 to Moeller et al; Europe Patent Publication NO 260 610 B1 by Moreller, A ET Al; Vasi, P (1992) ANNU. REV. IMMUNOL. 10: 411-452; Tracery, K j and Cervi, A (1994) Annu. REV. MED. 45: 491-503)

Therefore, in many human diseases, In order to reduce the harmful effect of over-expression of human tumor necrosis factor-alpha, the treatment strategy is designed to inhibit the bioactivity of TNF-a, for example, to use monoclonal antibody (adalimumab) to combine with tumor necrosis factor-alpha and to neutralize the bioactivity of TNF-a.

The adalimumab is approved by FDA to treat tumor necrosis factor-alpha related diseases such as ankylosing spondylitis, Crohn's disease, rheumatoid arthritis, psoriasis arthritis, ulcerative colitis, etc., and it has a significant curative effect, proving that over expression of the TNF-alpha does result as main factors of the above diseases.

The interleukin plays an important role in inflammatory response. Most human diseases are caused by chronic inflammation, which may affect joints, blood vessels or organs, and can even be fatal. Interleukin-1 (IL-1) is also called lymphocyte stimulating factor, which is one of the main driving cytokines of local and systemic inflammatory reactions. The IL-1 is mainly generated by activated mononuclear macrophages and mainly exists in two forms of IL-1 alpha and IL-1 beta, they bind to an interleukin-1 receptor (IL-1R) expressed on the cell surface to trigger the cascade reactions of pro-inflammatory medium, chemokine, and other cytokine. [11]

With the development and progress of medical science, more and more research data support that inflammation plays a certain role in tumor development, research shows that many malignant tumors appear in chronic inflammatory regions; and researches also show that the metastasis of tumor cells requires close cooperation among tumor cells, immune cells, inflammatory cells and stromal cells. [12,13] In addition, the deficiency in inflammation recovery process can be main cause in tumor invasion, progression and metastasis. [14,15] Various cells such as immune cells, nerve cells, endothelial cells and the like can secrete IL-1 beta, by combining with an IL-1 beta receptor, the related signal paths are mediated and activated. Finally, a large number of cell transcription factors are activated, including nuclear factor-kappa B to result in a series of consequences, for example, inflammatory response is activated to inhibit tumor immune response, so tumor growth, metastasis and invasion are promoted. [16-18]

Based on the above related mechanisms, IL-1 beta is considered to be a critical treatment target in an inflammatory disease or in promotion of tumor growth. IL-1 beta monoclonal antibodies are a class of novel anti-inflammation drugs that exert pharmacological effects on inflammatory cytokines, and treat inflammation and relieve diseases by blocking the binding of interleukin-1 beta to cell surface receptors.

At present, there are three monoclonal antibody targeting IL-1 beta therapies approved for clinical use, rilonacept in the treatment of gouty arthritis, anakinra in rheumatoid arthritis, kanakinumab has been demonstrated to reduce the mortality of myocardial infarction, stroke, and cardiovascular diseases of atherosclerosis patients [19-26], and reducing the risk of lung cancer and gout for patients as well. [27-31]

On top of that, medical experiments also demonstrate that IL-1 beta and TNF-alpha are also associated with the following diseases: diabetes, [32-35] diabetic retinopathy, [36-39] diabetic peripheral neuropathy, [40-41] myocarditis, [42-45] systemic lupus erythematosus, [46-50] osteoarthritis, [51-53] traumatic arthritis, [54-55] neuropathic pain, [55-59] cancerous pain, [60-64] pancreatic cancer, [65-66] liver cancer, etc. [67-68]

### SUMMARY OF THE INVENTION

Our researches discover that elapidae snakes including naja atra, Naja kaouthia, Bungarus multicinctus, Ophiophagus Hannah, Naja anchietae, Naja Nivea, Bungarus fasclatus, Dendroaspis polylepis and the like, their post-synaptic neurotoxin monomer molecules can inhibit or reduce the concentration of tumor necrosis factor-alpha (TNF-alpha) and interleukin 1 beta (IL-1 beta) in blood in an inflammation rat model, this discovery is reported for the first time.

The elapidae postsynaptic neurotoxin has a common three-finger structure and is also called three-finger toxin, and the active part is close to the middle finger tail end [59]. The three-finger structure is a multifunctional structure which has the common characteristic of adjusting acetylcholine and nicotinic acetylcholine receptors, three-finger toxin can reversibly and selectively combine with a nicotine acetylcholine subtype receptor, and the concentration of acetylcholine can be indirectly increased; more over the three-finger toxin can directly increase the concentration of acetylcholine by inhibiting acetylcholinesterase as well. [69-72] Acetylcholine is a neurosignal transmitter in the cholinergic anti-inflammatory pathway, and it's an agonist of nicotinic acetylcholine receptor subtype-alpha-7 which involved directly in the cholinergic anti-inflammatory pathway. Under the high-concentration of acetylcholine, the signal of “cholinergic anti-inflammatory pathway” is amplified, thus the capability of inhibiting inflammatory cytokines is also enhanced.

Therefore, the elapidea postsynaptic neurotoxin has the effect of inhibiting or reducing the expression of tumor necrosis factor-alpha (TNF-alpha) and interleukin 1 beta (IL-1 beta) in blood by adjusting the above “cholinergic anti-inflammatory pathway”, thus, this is a commonality of elapidae postsynaptic neurotoxin due to the common functional structure of three-finger toxin. However, the effect of inhibiting or reducing the expression of tumor necrosis factor-alpha (TNF-alpha) and interleukin 1 beta (IL-1 beta) in blood is need to be further studied by means of whether the “cholinergic anti-inflammatory pathway” acts as a solo mechanism or not.

Our studies find that these postsynaptic neurotoxin monomer molecules are the neurotoxins with the mature proteins or polypeptides of amino acid sequence (FASTA) as follows:

Postsynaptic neurotoxin of Naja atra
SEQ ID No.1
lechnqqssqtptttgcsggetncykkrwrdhrgyrtergcgcpsvkngieinccttdrcnn
SEQ ID No.2
mktllltllvvtivcldlgytlechnqqssqtptttgcsggetncykkrwrdhrgyrtergcgcpsvkngieinccttdrcnn
SEQ ID No.3
lechnqqssqtptttgcsggetncykkrwrdhrgyrtergcgcpivkngiesnccttdrcnn
Postsynaptic neurotoxin of Bungarus multicinctus
SEQ ID No.4
ivchttatspisavtcppgenlcyrkmwcdafcssrgkvvelgcaatcpskkpyeevtccstdkcnphpkqrpg
SEQ ID No.5
ivchttatipssavtcppgenlcyrkmwcdafcssrgkvvelgcaatcpskkpyeevtccstdkcnhppkrqpg
Postsynaptic neurotoxin of Naja anchietae
SEQ ID No.6
ircfitpdvtsqacpdgqnicytktwcdnfcgmrgkrvdlgcaatcptvkpgvdikccstdncnpfptrers
Postsynaptic neurotoxin of Naja Nivea
SEQ ID No.7
ircfitpdvtsqacpdghvcytkmwcdnfcgmrgkrvdlgcaatcpkvkpgvnikccsrdncnpfptrkr s
Postsynaptic neurotoxin of Ophiophagus hannah
SEQ ID No.8
tkcyktgdriiseacppgqdlcymktwcdvfcgtrgrvielgctatcptvkpheqitccstdncdphhkmlq
SEQ ID No.9
tkcyktgdriiseacppgqdlcymktwcdvfcgtrgrvielgctatcptvkpheqitccs
tdncnphpkmkq
SEQ ID No.10
tkcyitpdvksetcpdgenicytkswcdvfctsrgkridlgcaatcpkvkpgvdikccst
dncnpftpwkrh
SEQ ID No.11
tkcyvtpdatsqtcpdgqdicytktwcdgfcssrgkridlgcaatcpkvkpgvdikccst
dncnpfptwkrkh
SEQ ID No.12
tkcyvtpdvksetcpagqdlcytetwcvawctvrgkrvsltcaaicpivppkvsikccst
dacgpfptwpnvr
SEQ ID No.13
tkcyvtpdvksetcpagqdicytetwcdawctsrgkrvdlgcaatcpivkpgveikccst
dncnpfptwrkrp
Postsynaptic neurotoxin of Naja kaouthia
SEQ ID No.14
lechnqqssqapttktcsgetncykkwwsdhrgtiiergcgcpkvkpgvnInccrtdron n
SEQ ID No.15
lechnqqsiqtptttgcsggetncykkrwrdhrgyrtergcgcpsvkngieinccttdrcnn
SEQ ID No.16
mktllltllvvtivcldlgytlechnqqssqtptttgcsggetncykkrwrdhrgyrtergcgcpsvkngieinccttdrcnn
Postsynaptic neurotoxin of Dendroaspis polylepis
SEQ ID No.17
xicynhqsttrattksceenscykkywrdhrgtiiergcgcpkvkpgvgihccqsdkcny
SEQ ID No.18
ricynhqsttrattksceenscykkywrdhrgtiiergcgcpkvkpgvgihccqsdkcny
SEQ ID No.19
rtcnktfsdqskicppgenicytktwcdawcsrrgkivelgcaatcpkvkagvgikccstdnonlfkfgkpr
Postsynaptic neurotoxin of Bungarus fasclatus
SEQ ID No.20
riclnqqqstpedqptngqcyiktdcqnktwnthrgsrtdrgcgcpkvkpginlrccktd
kcne
SEQ ID No.21
Riclnqqssepqttetcpngedtcynktwnthrgsrtdrgcgcpkvkpginlrccktdkcnq

The amino acid sequences of the above elapidea postsynaptic neurotoxins are submitted separately in ASCII text file in the name of “SequencelistingCorrected”, created 2023-06-17, with size of 15 KB.

For the purpose of product manufacturing, as the postsynaptic neurotoxin monomer molecule from elapidae disclosed by the invention has a determined amino acid sequence, it can be produced through genetic engineering that solves the practical problem of scarcity of snake venom resources; even if we continue to process postsynaptic neurotoxin through separation and purification of natural snake venom, it is easier to control the quality and purity due to the determined amino acid sequence in the manufacturing process, which build up a necessary basis for the drug development of monomer from snake venom. Finally, the application of postsynaptic neurotoxin molecule can avoid the synergistic toxicity effect caused by a general snake venom mixture, for example, the postsynaptic neurotoxin molecule can avoid respiratory suppression due to the fact that pre-synaptic neurotoxin acts on the motor nerve synapse front film to block the release of acetylcholine causing paralysis of the skeletal muscle, but postsynaptic neurotoxin molecule doesn't, so improve the safety of the product in clinical uses.

The present invention is further described below with reference to the specific embodiments, however, the following embodiments are not intended to limit the present invention. Meanwhile, the equivalent substitutions in the art are all within the protection scope of the present invention.

### METHOD OF IMPLEMENTATION

### Example A: The Method for Specific Isolation and Extraction of Postsynaptic Neurotoxin SEQ ID NO 1

Mature protein or polypeptide of the current invention refers to the Chinese patent application publication number: CN 110090296A as an example.

### Example B: Method of Obtaining Bungarus multicinctus Postsynaptic Neurotoxin SEQ ID NO.4

The protein or polypeptide is produced by gene recombinant technology and is specified as follows:

1. Cloning of Recombinant Expression Vector

A DNA sequence is synthesized according to the gene of Bangarus postsynaptic neurotoxin (SEQ ID NO. 4) provided on GenBank, PCR amplification is carried out on a target DNA sequence, a sequence encoding the intestinal kinase recognition site, and an NDE I enzyme digestion site is introduced at the 5′ end of the upstream primer, and a stop codon and a BamHI enzyme digestion site are introduced at the 5′ end of the downstream primer.

The gene containing the postsynaptic neurotoxin SEQ ID No. 4 is amplified by using a PCR method and cloned into a PBS-T vector, and the constructed recombinant sub-PBS-T-Bangarus postsynaptic neurotoxin SEQ ID No. 4 shall be under examination with analysis and identification.

2. Protein Expression in E. coli.

The recombinant plasmid is transformed into the Escherichia coli expression vector pET15b, and the recombinant expression plasmid pET15b-postsynaptic neurotoxin SEQ ID No. 4 is constructed, and the correct recombinant transplanting to Escherichia coli BL21 (DE3)—LysS and examined under analysis and identification. The monoclonal antibody is inoculated into a 5G LB culture medium, cultured overnight at 37 DEG C., the day is inoculated into a 50 ml LB culture medium according to a ratio of 1:100, and the culture is shaken at 37 DEG C. until the OD600 nm is equal to 0.4-0.6.

3. Collection and Analysis of Expressed Products

The transformation is continued with 1 mmol/L of IPTG for 3 hours, the expression bacteria, which means the induced and transformed Escherichia coli BL21 (DE3), is centrifuged, the inclusion body is dissolved in the buffer solution, and the supernatant and the precipitate are subjected to SDS-PAGE electrophoresis detection after centrifugation and collection, and the target protein exists in the form of inclusion bodies.

4. Affinity and Purification of Expressed Products

The inclusion body after ultrasonic breaking is dissolved in a buffer solution (6 mol/L of guanidine hydrochloride, 20 mmol/L of Tris-HCl, ph 8.0, 0.5 mol/L of Nacl, 5 mmol/L imidazole); the buffer solution is purified through a nickel-NTA column affinity chromatography, specifically, the buffer solution containing 20 mmol/L imidazole is washed to a baseline before loading, and finally, the buffer solution containing 300 mol/L imidazole is used for elution. The enterokinase is used to cut for obtaining the postsynaptic neurotoxin SEQ ID No. 4 protein.

5. Renaturation of Expressed Product

The eluted protein is dialyzed with 6 mol/L guanidine hydrochloride, 0.1 mol/L Tris-HCl—HCl Ph8.0, 0.01 mol LEDTA, 0.1 mmol/L PMSF, 10 mmol/L DTT buffer solution, the concentration of DTT and guanidine hydrochloride in the buffer solution is gradually decreased, and then the buffer solution is dialyzed with 10-fold volume of 0.1 mol/L Tris-HCL Ph-8.0, 5 μmol/L CuSo4, and 20% glycerol. An RP-HPLC method is used for detecting the renaturation result, and through the comparison of retention time with a standard sample, the renaturation substance is identified, and the renaturation product is refrigerated and stored.

6. Amino Acid Sequence Determination

The peptide fragment coverage rate and the Edman degradation method are used for the analysis of the obtained postsynaptic neurotoxin, and the measured SEQ ID No. 4 sequence is compared with the amino acid sequence of the neurotoxin in the protein library, and after the confirmation of the consistency of the sequence, then the postsynaptic neurotoxin is used for the anti-inflammation test in the next step.

### Example C: An Experiment of Anti-Inflammation Properties of Elapidae Postsynaptic Neurotoxin Monomer Molecule

1. Animals and Grouping

140 Wistar rats (200-240 g) are randomly divided into 14 groups, and each group is 10. The preparation method comprises the following steps: (group 1) only receiving sterile normal saline (0.95% of sodium chloride) (control group); (group 2) carrageenan inflammation modeling+oral normal saline 10 ml/kg (inflammation group); (Group 3) Carrageenan Inflammation modeling+oral Naja atra postsynaptic neurotoxin (SEQ ID NO 1) 200 μg/kg prepared into a liquid state and continuous intragastric administration performed; (Group 4) Carrageenan Inflammation modeling+oral Naja atra postsynaptic neurotoxin (SEQ ID NO 1) 800 μg/kg prepared into a liquid state and continuous intragastric administration performed. (Group 5-14) The postsynaptic neurotoxin of Bungarus multicinctus, Ophiophagus Hannah, Naja kaouthia, Dendroaspis polylepis, Bungarus fasclatus, through the same administration model, that means carrageenan inflammation modeling+oral administration of 200 μg/kg and 800 μg/kg two doses respectively and continuously intragastric administration, so that the rat is divided into 14 groups.

2. Animal Inflammation Modeling

Except for the illumination group, 0.1 ml of sterile normal saline+carrageenan (CG, 1%) is injected into the rest 13 groups through pleural cavities for inflammation modeling. The preparation method comprises the following steps: 1 hour before molding, orally administration of normal saline or different types of postsynaptic neurotoxin with different doses respectively in each group, 6 hours after the carrageenan injection, collecting rat tail vein blood to detect the concentration of IL-1 beta and TNF-alpha.

3 Measurement of Tumor Necrosis Factor Alpha (TNF-Alpha) and Interleukin 1 Beta (IL-1 Beta)

The serum of the collected rat tail vein blood is separated and collected by using a commercial ELISA assay kit, and biochemical evaluation is carried out on the concentration of the tumor necrosis factor alpha (TN F-alpha) and interleukin 1 beta (IL-1 beta) according to the instructions of experimental step of the kit's manufacturer.

4. Experimental Results

TABLE 1
is a comparison of the blood average concentration of tumor
necrosis factor-alpha (TNF-alpha) and interleukin 1 beta
(IL-1 beta) among the rat control group, the inflammation
group and six postsynaptic neurotoxin treatment groups.

| | IL-1β | TNF-a |
|---|---|---|
| n = 10 | (ng/L, x ± s) | (ng/L, x ± s) |
| Saline Control Group | 35.20 ± 4.78 | 42.70 ± 5.93 |
| Inflammation Group | 52.40 ± 6.74 | 61.30 ± 7.86 |
| SEQ ID No1(200 μg/kg) | 45.40 ± 4.95* | 53.70 ± 6.32* |
| SEQ ID No1(800 μg/kg) | 41.30 ± 4.31*** | 50.60 ± 5.89** |
| SEQ ID No4(200 μg/kg) | 46.70 ± 5.12* | 52.76 ± 6.43* |
| SEQ ID No4(800 μg/kg) | 39.80 ± 5.01*** | 48.80 ± 5.41*** |
| SEQ ID No9(200 μg/kg) | 44.50 ± 4.97** | 54.80 ± 5.01* |
| SEQ ID No9(800 μg/kg) | 41.70 ± 4.23*** | 51.20 ± 5.76** |
| SEQ ID No14(200 μg/kg) | 43.50 ± 5.43** | 53.01 ± 6.87* |
| SEQ ID No14(800 μg/kg) | 38.20 ± 4.35*** | 49.50 ± 5.12** |
| SEQ ID No17(200 μg/kg) | 46.02 ± 4.53 * | 52.20 ± 6.35 * |
| SEQ ID No17(800 μg/kg) | 43.80 ± 3.98 ** | 50.10 ± 6.02 ** |
| SEQ ID No20(200 μg/kg) | 44.10 ± 4.32 * | 52.82 ± 6.55 * |
| SEQ ID No20(800 μg/kg) | 40.80 ± 3.78 ** | 47.81 ± 5.72 *** |

Experimental results show that total 12 postsynaptic neurotoxin treatment groups, in each treatment group, the average blood concentration of tumor necrosis factor-alpha (TNF-alpha) and interleukin 1 beta (IL-1 beta) of is lower than that of the inflammation group, and it shows a significant difference and dose-dependent effect between the treatment group and inflammation group. *representing P<0.05, **representing P<0.01, ***representing P<0.001.

### REFERENCE

1. Tracey K J. The inflammatory reflex. Nature, 2002, 420(6917): 853-859.
2. Tracey, K. J. et al. Shock and tissue injury induced by recombinant human cachectin. Science 234, 470-474(1986).
3. Wang, H. et al. HMG-1 as a late mediator of endotoxin lethality in mice. Science 285, 248-251(1999).
4. Tracey, K. J. et al. Anti-cachectin/TNF monoclonal antibodies prevent septic shock during lethal bacteraemia. Nature 330, 662-664(1987).
5. Tracey, K. J. & Cerami, A. Tumor necrosis factor: a pleiotropic cytokine and therapeutic target. Annu. Rev. Med. 45, 491-503 (1994).
6. WANG, Bojie et al., Clinical Application of Cholinergic Anti-Inflammatory Pathway, “Anesthesia and Monitoring Forum” 2005
7. Lyudmila V. Borovikova et al. Vagus nerve stimulation attenuates the systemic inflammatory response to endotoxin. Nature 2000 405: 458-462
8. Zheng Cheng et al., Research on protective effect of cholinergic anti-inflammatory pathway in mouse viral myocarditis. Master's Theses of Wenzhou Medical University 2014
9. WANG, Bojie et al., Research Progress of Cholinergic Anti-Inflammatory Pathway. International Anesthesiology and Resuscitation Magazine 2006 (04)
10. Liu Suiliang et al., Research Progress of Cholinergic Anti-Inflammatory Pathway. International anesthesiology and resuscitation magazine 2007 Jan. 18
11. Dinarello C A, Simon A, van der Meer J W. Treating inflammation by blocking interleukin-1 in a broad spectrum of diseases. Nat Rev Drug Discov. 2012; 11(8): 633-652. doi: 10.1038/nrd3800.
12. Armstrong H, et al. Cancers (Basel) 2018; 10. pii: E83.
13. Grivennikov S I, Greten F R, Karin M. Immunity, inflammation, and cancer cell. 2010; 140(6): 883-899. doi: 10.1016/j.cell.2010.01.025.
14. Apte R N, Voronov E. Immunotherapeutic approaches of IL-1 neutralization in the tumor microenvironment. J Leukoc Biol. 2017; 102(2): 293-306. doi: 10.1189/jlb.3MR1216-523R.
15. Porta C, Larghi P, Rimoldi M, et al. Cellular and molecular pathways linking inflammation and cancer. Immunobiology. 2009; 214(9-10): 761-777. doi: 10.1016/j.imbio.2009.06.014.
16. Dhimolea E. Canakinumab. MAbs. 2010; 2(1): 3-13. doi: 10.4161/mabs.2.1.10328.
17. Gram H. Preclinical characterization and clinical development of ILARIS (canakinumab) for the treatment of autoinflammatory diseases. Curr Opin Chem Biol. 2016; 32: 1-9. doi: 10.1016/j.cbpa.2015.12.003.
18. Multhoff G, Molls M, Radons J. Chronic inflammation in cancer development. Front Immunol. 2012; 2: 98. Published 2012 Jan. 12. doi: 10.3389/fimmu.2011.00098
19. Jiao Yabin et al., The expression of L-1 beta, TNF alpha, IL-10 and IL-10 R of aorta of rat with atherosclerosis and ginkgo leaf extract function. Journal of Second Military Medical University (Journal of Second Military Medical University) 2005
20. Shubair, M. K. IL-1 I3 level in Sudanese patients with atherosclerotic coronary heart disease. Int J Med Biomed Res 2012; 1(1): 73-7873.
21. Qamar, Arman et al., Effect of interleukin 1 I3 inhibition in cardiovascular disease. Current opinion in lipidology, 12/2012, Volume 23, Issue 6.
22. Khan, Razi et al., Examining the Role of and Treatment Directed at IL-1 I3 in Atherosclerosis. Current Atherosclerosis Reports, November 2018, Volume 20, Issue 11.
23. Hsue, Priscilla Y et al., IL-1 I3 Inhibition reduces Atherosclerotic Inflammation in HIV Infection. Journal of the American College of Cardiology, December 2018, Volume 72, Issue 22.
24. WeiJinjin et al. L0X-1, IL-1 Beta in Human Porridge Hardened Crown Tongzi and FIG., Luxury Congee Hardened and Spread.
25. ZHANG et al. The change of serum IL-1 beta, IL-6 and coronary macrophage CD68 of atherosclerosis rat “Chinese Senile Science”-2013
26. Qiao chunchun et al., The expression of serum IL-1 beta and IL-1 RA of atherosclerosis rats and the influence of simvastatin on it's expression The “Bengbu Medical College” 2012
27. E. Ridker P M et al., on behalf of the CANTOS Trial Group. Antiinflammatory therapy with canakinumab for atherosclerotic disease. N Engl J Med 2017; 377: 1119-31.
28. Harrington R A. Targeting Inflammation in Coronary Artery Disease. N Engl J Med 2017; 377: 1197-8.
29. Ridker P M et al., on behalf of the CANTOS Trial Group. Effect of interleukin-1 I3 inhibition with canakinumab on incident lung cancer in patients with atherosclerosis: exploratory results from a randomised, double-blind, placebo-controlled trial. Lancet 2017; 390: 1833-42.
30. Ridker P M et al., on behalf of the CANTOS Trial Group. Relationship of C-reactive protein reduction to cardiovascular event reduction following treatment with canakinumab: a secondary analysis from the CANTOS randomised controlled trial. Lancet 2018; 391: 319-28.
31. Schlesinger Naomi et al., Canakinumab reduces the risk of acute gouty arthritis flares during initiation of allopurinol treatment: Annals of the Rheumatic Diseases, July 2011, Volume 70, Issue 7
32. A Zolingue et al, The Chinese patent publication number CN101616690A for treating the diabetes by the method for treating the IL-1 beta-related diseases is treated by a method for treating IL-1 beta-related diseases.
33. Doody, Natalie E et al., The Role of TLR4, TNF-α and IL-1β in Type 2 Diabetes Mellitus Development within a North Indian Population. Annals of Human Genetics, July 2017, Vol 81, Issue 4
34. Jiang, Zhu-Ling et al., Study of TNF-α, IL-1β and LPS levels in the gingival crevicular fluid of a rat model of diabetes mellitus and periodontitis. Disease markers, 2013, Vol 34, Issue 5
35. Ortis, F et al., Differential usage of NF-κB activating signals by IL-1β and TNF-α in pancreatic beta cells. FEBS Letters, April 2012, Volume 586, Issue 7
36. S Doganay, Comparison of serum NO, TNF-α, IL-1 I3, sIL-2R, IL-6 and IL-8 levels with grades of retinopathy in patients with diabetes. Eye, March 2002, Volume 16, Issue 2.
37. Hanqin. The detection and clinical significance of peripheral blood IL-1 beta and TNF-alpha of diabetic retinopathy patients. Chinese Experimental Diagnostics, 20 Apr. 2016, vol. 20, no. 4
38. Youhui et al. The significance of detection serum IL-1 beta and TNF-alpha of patients with diabetic retinopathy accompanied with hyperuricemia. International Journal of Laboratory Medicine. 2018 DOI: CNKI: Sun: GWSQ. 0.2018-13-013
39. Chen Jinguang. Clinical significance of analysis and detection of peripheral blood IL-1 beta and TNF-alpha level of diabetic retinopathy patients. Medical Theory and Practice, no. 6, 2017 Zhou Weiya. The mediation of IL-1 beta and TNF-alpha in diabetic peripheral neuropathy. Chinese Journal of Immunology, 2003, vol. 9, no. 9
40. Zhouweiya. The mediation of IL-1 beta and TNF-alpha in diabetic peripheral neuropathy. Chinese Journal of Immunology, 2003, vol. 9, no. 9
41. LanXinlian et al, Influences of Berberine on IL-1, TNF-alpha and NO of diabetic II peripheral neuropathy rats. [J]. SHAANXI MEDICAL MAGAZINE. 2009 (04)
42. Songyin. Research of effect of IL-1 and it's family members on myocarditis. modern biomedical progress 2016 and 16 vol. 35
43. Pizhiyu. Measurement and significance of serum nitric oxide, IL-1 beta and tumor necrosis factor (TNF) content of 3-pichizia viral myocarditis patients. Chongqing Medicine, 28 Jun. 2008, volume 11
44. Chen, Zhijian et al., Expression and significance of IL-1 beta and TNF-alpha in mouse giant cell viral myocarditis tissue. In 2005, Shandong Medicine 2005
45. Tang Xingsan et al., Expression of IL-1β and TNF-α in MCMV myocarditis and its role. Journal of Huazhong University of Science and Technology[Medical Sciences] volume 25, pages 254-256(2005).
46 Liu et al., Expression and measurement of IL-17 and TNF-alpha in serum of systemic lupus erythematosus patients. Qingdao Medical Health, 2020, no. 1
47, Zhao et al., peripheral blood IL-17, TNF-alpha and INF-gamma expression and clinical significance of systemic lupus erythematosus patients. The medicine magazines of Liberation Army 2017, vol. 29, no. 6
48. Vinod Umare et al., Effect of Proinflammatory Cytokines (IL-6, TNF-α, and IL-1 I3) on Clinical Manifestations in Indian SLE Patients. Mediators Inflamm. 2014 Dec. 7.
49. Sabry A1 et al., Proinflammatory cytokines (TNF-alpha and IL-6) in Egyptian patients with SLE: its correlation with disease activity. Cytokine. 2006 August; 35(3-4): 148-53.
50. Wozniacka, A et al., The influence of antimalarial treatment on IL-1β, IL-6 and TNF-α mRNA expression on UVB-irradiated skin in systemic lupus erythematosus. British Journal of Dermatology, 11/2008, Volume 159, Issue 5.
51. P. F., Zangerle et al; Direct stimulation of cytokines (IL-1β, TNF-α, IL-6, IL-2, IFN-γ and GM-CSF) in whole blood: II. Application to rheumatoid arthritis and osteoarthritis. [symbol] Cytokine [symbol] , 1992.
52. Alikhanov, B A et al; Efficiency and Tolerability of Artrofoon (Ultralow Doses and Antibodies to TNF-α) in Osteoarthrosis, Bulletin of experimental biology and medicine, September 2009, Volume 148, Issue 3.
53. Serum levels of the bone turnover markers dickkopf-1, osteoprotegerin, and TNF-α in knee osteoarthritis . . . Clinical rheumatology, October 2017, Volume 36, Issue 10.
54 Tang et al., Lentivirus mediating RNAi silencing IL-1 beta and TNF-alpha to treat post-traumatic arthritis. [Master Paper] Nanchang University 2016
55. Qiu et al, The level change and significance of TNF-alpha, IL-6 and nitric oxide in the joint liquid of traumatic arthritis patients. Chinese bone and joint injury magazine.
56. Ma et al., Research on the Effect of TNF-alpha in Neuropathic Pain. EXTERNAL MEDICAL TREATMENT
57. Research Progress of Inflammatory Factor TNF-alpha, IL-1 beta, IL-6 in neuropathic pain. China Traditional Chinese Medicine Journal 2017, 19
58. Yang, Qing-Qing et al; Red nucleus IL-6 mediates the maintenance of neuropathic pain by inducing the productions of TNF-α and IL-1β. Neuropathology, August 2020, Volume 40, Issue 4
59. Zhou, Zhigang et al; A novel cell-cell signaling by microglial transmembrane TNFα with implications for neuropathic pain. Pain (Amsterdam), November 2010, Volume 151, Issue 2
60. Mao, Mao; Li, et al; Aitongxiao improves pain symptoms of rats with cancer pain by reducing IL-1, TNF-α, and PGE2. International journal of clinical and experimental pathology, 2021, Volume 14, Issue 1
61. Salvo, Elizabeth et al; TNFα promotes oral cancer growth, pain, and Schwann cell activation. Scientific reports, January 2021, Volume 11, Issue 1
62. Li, Qingsong et al; Epigallocatechin-3-gallate attenuates bone cancer pain involving decreasing spinal Tumor Necrosis Factor-α. International immunopharmacology, December 2015, Volume 29, Issue 2
[63. Gu, Xiaoping et al; Intraperitoneal Injection of Thalidomide Attenuates Bone Cancer Pain and Decreases Spinal Tumor. Molecular pain, October 2010, Volume 6, Issue 1
64. Mazaki, Ai et al; Tumor Necrosis Factor-α Produced by Osteoclasts Might Induce Intractable Pain in a Rat Spinal Metastasis Model of Breast Cancer. Spine surgery and related research, July 2019, Volume 3, Issue 3
65. Liu et al., the effect of TNF-alpha in the development of experimental pancreatic cancer models. China General Surgery Journal, 2008.781-784
66. Li Xiuxiu et al., serum SIL-2 R and TNF level detection and significance before and after operation of pancreatic cancer patients. Tumor research and clinic, 1996 (008) 001
67. Luyan et al., the double perfusion intervenes in the treatment of the changes in serum TNF-alpha, SDI-2 R and VEGF levels of liver cancer patients. Journal of Utility Liver Disease, 2010
68 WangMingyu et al, serum TNF-alpha level detection of primary liver cancer patients and clinical significance of the serum TNF-alpha level detection. JOURNAL OF UTILITY CANCER 2000
69. J. White et al, 1996 Snake Neurotoxin. Human Toxicology, 1996.
70. Pascale Marchot et al, The three-finger toxin fold: a multifunctional structural scaffold able to modulate cholinergic functions. Journal of neurochemistry, Vol 142, issue S2, 2017.
71. Nirthanan S1 et al., Three-finger alpha-neurotoxins and the nicotinic acetylcholine receptor, forty years on. J Pharmacol Sci. 2004 January; 94(1): 1-17.
72. Victor I et al., Three-finger snake neurotoxins and Ly6 proteins targeting nicotinic acetylcholine receptors: pharmacological tools and endogenous modulators. Trends in Pharmacological Sciences. Volume 36, Issue 2, February 2015, Pages 109-123

1. A method for treating excessive expression of tumor necrosis factor-α, (TNF-α) and interleukin-1β, (IL-1β) in a mammal. Said method comprises administering to a mammal in need thereof a pharmaceutical composition of a therapeutically effective amount of an Elapidae postsynaptic neurotoxin monomer molecule, (SEQ ID No.1-21) and a pharmaceutically acceptable carrier base, for use in inhibiting or reducing the concentration of tumor necrosis factor-α, (TNF-α) and interleukin-1β, (IL-1β) in blood and human body.

2. A method for treating excessive expression of tumor necrosis factor-α, (TNF-α) and interleukin-1β, (IL-1β) in a mammal. Said method comprises administering to a mammal in need thereof a pharmaceutical composition of a therapeutically effective amount of an Elapidae postsynaptic neurotoxin monomer molecule, (SEQ ID No.1-21) and a pharmaceutically acceptable carrier base, for use in treating or preventing the disease associated with excessive expression of tumor necrosis factor-α, (TNF-α) and/or interleukin-1β, (IL-1β).

3. The disease associated with excessive expression of tumor necrosis factor-α, (TNF-α) and/or interleukin-1β, (IL-1β) of claim (2), wherein it refers to Rheumatoid arthritis.

4. The disease associated with excessive expression of tumor necrosis factor-α, (TNF-α) and/or interleukin-1β, (IL-1β) of claim (2), wherein it refers to rheumatic arthritis.

5. The disease associated with excessive expression of tumor necrosis factor-α, (TNF-α) and/or interleukin-1β, (IL-1β) of claim (2), wherein it refers to gouty arthritis.

6. The disease associated with excessive expression of tumor necrosis factor-α, (TNF-α) and/or interleukin-1β, (IL-1β) of claim (2), wherein it refers to traumatic arthritis.

7. The disease associated with excessive expression of tumor necrosis factor-α, (TNF-α) and/or interleukin-1β, (IL-1β) of claim (2), wherein it refers to osteoarthritis arthritis.

8. The disease associated with excessive expression of tumor necrosis factor-α, (TNF-α) and/or interleukin-1β, (IL-1β) of claim (2), wherein it refers to ankylosing spondylitis.

9. The disease associated with excessive expression of tumor necrosis factor-α, (TNF-α) and/or interleukin-1β, (IL-1β) of claim (2), wherein it refers to diabetes.

10. The disease associated with excessive expression of tumor necrosis factor-α, (TNF-α) and/or interleukin-1β, (IL-1β) of claim (2), wherein it refers to diabetic neuropathy.

11. The disease associated with excessive expression of tumor necrosis factor-α, (TNF-α) and/or interleukin-1β, (IL-1β) of claim (2), wherein it refers to diabetic retinopathy.

12. The disease associated with excessive expression of tumor necrosis factor-α, (TNF-α) and/or interleukin-1β, (IL-1β) of claim (2), wherein it refers to systemic lupus erythematosus.

13. The disease associated with excessive expression of tumor necrosis factor-α, (TNF-α) and/or interleukin-1β, (IL-1β) of claim (2), wherein it refers to neuropathic pain.

14. The disease associated with excessive expression of tumor necrosis factor-α, (TNF-α) and/or interleukin-1β, (IL-1β) of claim (2), wherein it refers to cancer pain.

15. The disease associated with excessive expression of tumor necrosis factor-α, (TNF-α) and/or interleukin-1β, (IL-1β) of claim (2), wherein it refers to myocarditis.

16. The disease associated with excessive expression of tumor necrosis factor-α, (TNF-α) and/or interleukin-1β, (IL-1β) of claim (2), wherein it refers to pancreatic cancer.

17. The disease associated with excessive expression of tumor necrosis factor-α, (TNF-α) and/or interleukin-1β, (IL-1β) of claim (2), wherein it refers to liver cancer.

18. The Elapidae postsynaptic neurotoxin of claim (2), wherein it is an Elapidae postsynaptic neurotoxin polypeptide having the amino acid sequence of SEQ ID No.1 to SEQ ID No.21, or an Elapidae neurotoxin polypeptide homologs having 70% or more homology with the Elapidae neurotoxin polypeptide of SEQ ID No.1 to SEQ ID No.21, and the biological function of the Elapidae postsynaptic neurotoxin polypeptide homologs is the same as or similar to that of the Elapidae neurotoxin polypeptide of the amino acid sequence ID No. 1 to SEQ ID No. 21.

19. The Elapidae postsynaptic neurotoxin polypeptides or Elapidae postsynaptic neurotoxin polypeptides homologs of claim (18), wherein it's further characterized in that they are isolated from natural snake venoms, or synthesized from chemical polypeptides, or obtained from prokaryotic or eukaryotic hosts using recombinant technology such as Bacteria, yeast, higher plants, insects and mammalian cells.

20. The method of claim (2) includes intravenous, intramuscular, subcutaneous, intra-articular, oral, sublingual, nasal, rectal, topical, intradermal, intraperitoneal, intrathecal, or transdermal administration, and the dose of the Elapidae postsynaptic neurotoxin includes from 1 μg/Kg to 2 mg/kg each time.
